(19) United States
(12) Patent Application Publication
(10) Pub. No.: US 2018/0000744 A1
(43) Pub. Date: Jan. 4, 2018

(54) NANOENCAPSULATED COMPOSITIONS

(71) Applicant: Georgia Southern Research and Service Foundation, Inc., Statesboro, GA (US)

(72) Inventors: Mujibur Rahman Khan, Augusta, GA (US); Sakib Iqbal, Statesboro, GA (US)

(21) Appl. No.: 15/639,215

(22) Filed: Jun. 30, 2017

Related U.S. Application Data
(60) Provisional application No. 62/356,838, filed on Jun. 30, 2016.

(51) Int. Cl. A61K 9/51; A61K 31/513; B05D 1/04

(52) U.S. Cl. CPC A61K 9/5146; B05D 1/04; A61K 9/5138; A61K 9/5192; A61K 31/513

(57) ABSTRACT

Disclosed herein are nano-encapsulated compositions and methods of preparing the same. The compositions may be obtained through electrospray and electrospin technologies. The nano-encapsulated composition exhibit improved pharmacokinetic properties.

### CROSS-REFERENCE TO RELATED APPLICATION

This application claims the benefit of U.S. Provisional Application 62/356,838, filed on Jun. 30, 2016, the contents of which are incorporated by reference in its entirety.

### FIELD OF THE INVENTION

The invention relates to nanomaterials for drug delivery, and methods of making the same using electrospinning and electrospraying processes.

### BACKGROUND

Electrospray is a technique for aerosolizing a liquid. Liquid is supplied through a capillary and a high voltage is applied to the tip of the capillary. Apart from the tip there is a plate having low voltage. The high potential at the tip results in the formation of a Taylor cone, whereby a liquid jet is emitted through the apex of the cone. The jet rapidly forms into droplets as a result of Coulombic repulsion in the jet.

Related to electrospray is electrospinning, in that a voltage source is connected between the tip of a capillary and a collector plate. A Taylor cone forms due to the combination of Coulombic repulsion and surface tension. If the liquid is sufficiently viscous, the emitted liquid jet does not break up, and instead deposits as a thin fiber on the plate. Judicious selection of polymer, solvent, field strength and other parameters enable the production of nanometer scale fibers.

Core-shell fibers can be obtained by a modification to the electrospinning technique designated coaxial electrospinning. The substance intended for the core is dissolved in a first solution, and the substance intended for the shell is dissolved in a second solution. The first solution is dispensed through the nozzle while the second solution is dispensed through a concentric ring surrounding the nozzle. The two solutions must have sufficient viscosity to be pulled into the Taylor cone to form the fibers. Historically, the two solutions have been immiscible with each other, to prevent the mutual diffusion that can occur in the Taylor cone as well as during stretching.

Nanomaterials such as nanoparticles and nanofibers have been the subject of substantial biomedical research over the last two decades, especially as carriers for drug delivery systems, for instance either as implantable, transdermal or fast dissolving systems. Selective delivery of nanoparticles and nanofibers directly to cancerous cells or tumors could mitigate many of the harmful side effects of chemotherapy. Furthermore, it has been reported that certain nanoparticles may selectively cross biological barriers, for instance the blood brain barrier. Nanoparticles may also be able to selectively access tumor cells on the premise that leaky vasculature present in tumor tissue may admit appropriately sized particles. This observation has been designated the Enhanced Permeability and Retention effect (“EPR effect”), which has been demonstrated with thermal ablation of gold nanoparticles.

There remains a need for nanomaterials like nanofibers and nanoparticles with improved properties, for instance as drug carriers with improved release characteristics and/or increased potency. There is a need for improved methods of preparing nanofibers and nanoparticles with high degrees of uniformity.

### SUMMARY

Disclosed herein are uniform core/shell nanomaterials containing at least one pharmaceutically active ingredient. In some embodiments, the core/shell nanomaterial can be a nanofiber, wherein the pharmaceutically active ingredient is in the core component, and the shell includes at least one biodegradable, biocompatible polymer. In other embodiments, the core/shell nanomaterial can include a drug, which may be crystalline, encapsulated in a biodegradable, biocompatible polymer.

The core/shell materials can be prepared using a modified electrospinning process, whereas the nanocrystalline drug can be prepared using a modified electrospraying process.

The details of one or more embodiments are set forth in the descriptions below. Other features, objects, and advantages will be apparent from the description and from the claims.

### BRIEF DESCRIPTION OF THE FIGURES

FIG. 1, Panel A includes a depiction of SEM images of neat PCL NFs exhibited thin fibrous network of narrow size distribution ranging from 80-120 nm. Neat PCL NFs exhibited no presence of beads or bundles of fibers.

FIG. 1, Panels B-F includes a depiction SEM images of nanofibers having fluorouracil core at different magnifications. In Panel B, large beads of 0.3-1.2 μm are visible within the fiber network. Panel D depicts nanofibers that are randomly distributed with many non-uniform surface features with lots of FU particles attached to the surface. Diameter of nanofibers was distributed between 120-350 nm at average diameter of 238 nm and standard deviation of 92 nm.

FIG. 2 includes a depiction of SEM images of uniform core/shell nanofibers. The fibers are characterized by the absence of large beads or irregular surfaces. Panels A and B depict a nanofiber having an average diameter of 170 nm (110-270 nm), with a standard deviation of 41. Panels C and D depict a nanofiber having an average diameter of 200 nm (120-350), with a standard deviation of 57.5. Panels E and F depict a nanofiber having an average diameter of 220 nm (140-400 nm), with a standard deviation of 53.8.

FIG. 3 includes a depiction of the in vitro release of fluorouracil from PCL nanofibers.

FIG. 4 includes a depiction of cell viability for PC3 cells in PCL nanofibers.

FIG. 5 includes a depiction of in vitro cytotoxicity of neat fluorouracil and core/shell nanofibers having fluorouracil.

FIG. 6 includes a depiction of prostatic cancer cells treated with fluorouracil loaded core/shell nanofibers.

FIG. 7 includes a depiction of SEM images of paclitaxel loaded core/shell nanofibers. The average diameter of the fibers was 46.8 nm (22-90 nm) with a standard deviation of 17.2.

FIG. 8 includes a depiction of diameter distribution of paclitaxel loaded core/shell nanofibers.

FIG. 9 includes a depiction of human breast cancer cells implanted with either neat PCL nanofibers or paclitaxel containing nanofibers over a period of 24, 48 and 72 hours.

FIG. 10, Panel A includes a depiction of fluorouracil nanocrystals. FIG. 10, Panels B and C include depictions of fluorouracil nanocrystals encapsulated in PCL.

FIG. 11 depicts an energy dispersive x-ray spectrum of NFs B2 and C2.

FIG. 12 depicts a TEM image FU/cisplatin encapsulated nanofiber. The fiber exhibited drug core of app 80-90 nm and polymer sheath of 30-40 nm thickness.

FIG. 13 depicts the correlation between drug encapsulation efficiency and effective applied voltage.

FIG. 14 depicts the correlation between sheath/core flow ratio and diameter.

FIG. 15 depicts the correlation between drug release and pH.

FIG. 16 depicts cell viability of MDA-MB-231 triple negative breast cancer cells determined by WST assay. From left to right, the represent control, PCL only, PCL plus paclitaxel, PCL plus 5-FU, PCL plus paclitaxel, plus 5-FU

FIG. 17 depicts SEM images of nanocrystalline HET-0016

FIG. 18 depicts images of nanocrystalline FU.

FIG. 19 depicts SEM images FU loaded PCL nanoparticles processed through emulsion solvent evaporation method.

FIG. 20 depicts SEM images Paclitaxel loaded PCL nanoparticles processed through electrospraying of Paclitaxel and PCL solution using different solvents system and parameters.

FIG. 21 depicts an SEM image of electrosprayed particles of PCL-PEG-NH₂.

FIG. 22 depicts SEM image of PCL-PEG-NH₂Nanoparticle formation less than 50 nm

FIG. 23 depicts SEM image of 5 FU loaded PCL nanoparticles less than 100 nm

FIG. 24 depicts the chemical reaction between PCL-PEG-NH₂ and a dye.

FIG. 25 depicts: (a) SEM image of electrosprayed HET-0016 nanocrystal (b) (c) SEM image of electrosprayed HET-0016 drug nanocrystals after 1 hour sonication at different magnification

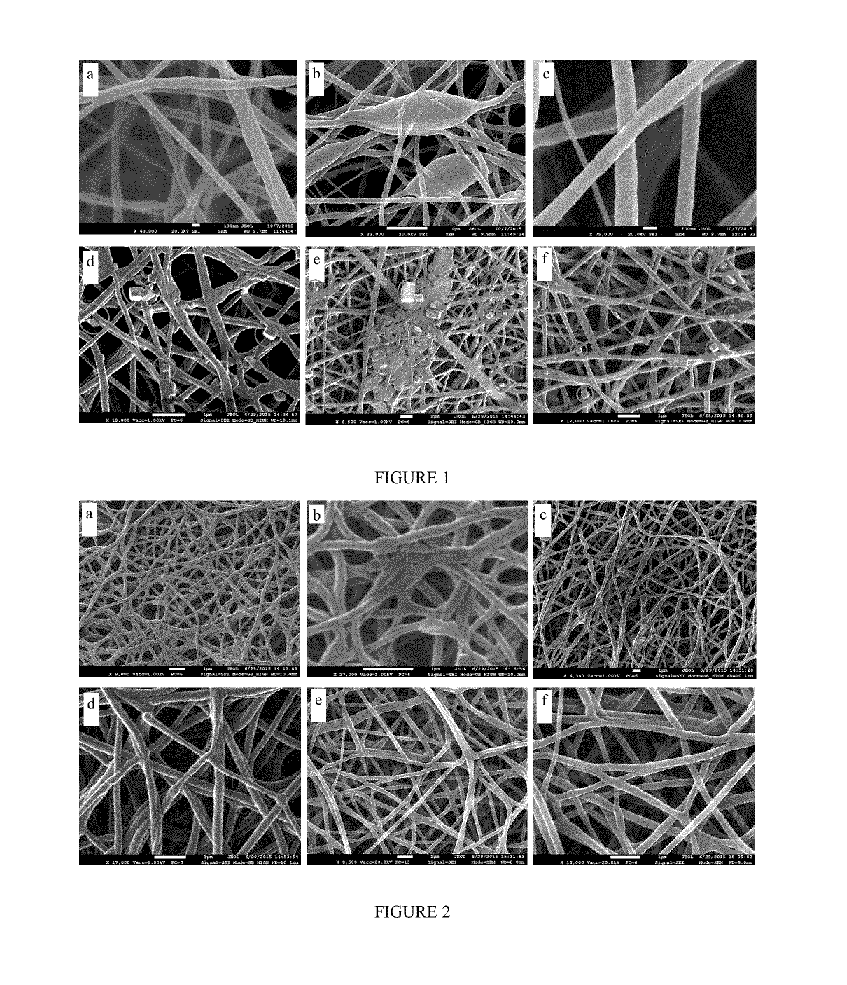

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes—from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Disclosed herein are uniform core/shell nanomaterials containing at least one pharmaceutically active ingredient. In some embodiments, the core/shell nanomaterial can be a nanofiber, wherein the pharmaceutically active ingredient is in the core component, and the shell includes at least one biodegradable, biocompatible polymer. In other embodiments, the core/shell nanomaterial can include a nanocrystalline drug encapsulated in a biodegradable, biocompatible polymer.

The core/shell nanomaterials can be characterized by at least one diameter (d). For embodiments in which the nanomaterial is a nanofiber, (d) refers to the average diameter perpendicular to the longitudinal axis. For embodiments in which the nanomaterial is a nanocrystalline drug encapsulated in a polymer, (d) refers to the average particle diameter of the drug and polymer together. The core/shell nanomaterials described herein can have at least one diameter (d) having a length no greater than about 1,000 nm, no greater than about 900 nm, no greater than about 800 nm, no greater than about 700 nm, no greater than about 600 nm, no greater than about 500 nm, no greater than about 400 nm, no greater than about 300 nm, no greater than about 200 nm, no greater than about 100 nm, no greater than about 75 nm, no greater than about 50 nm or no greater than about 25 nm. In certain embodiments, the core/shell nanomaterials described herein can have at least one diameter (d) from about 10-1,000 nm, about 10-900 nm, about 10-800 nm, about 10-700 nm, about 10-600 nm, about 10-500 nm, about 10-400 nm, about 10-300 nm, about 10-200 nm, about 10-100 nm, about 100-1,000 nm, about 100-750 nm, about 100-500 nm, 100-400 nm, 100-300 nm, about 100-250 nm, about 100-200 nm, about 25-200 nm, about 50-200 nm, or about 50-100 nm.

The core/shell nanomaterials described herein can be characterized by a high degree of uniformity. As used herein, the term “uniform” refers to a narrow distribution of fiber diameter (d) along the length of the fiber or a narrow distribution of particle sizes. The distribution can be characterized by the standard deviation of the diameter (d) along the length of the fiber. In some embodiments, the nanomaterial can be characterized by a standard deviation diameter (d) is no greater than 25%, 20%, 10%, 5%, 2.5% or 1% of the average diameter (d).

The core/shell nanomaterial can be characterized by a high encapsulation efficiency. As used herein, encapsulation efficiency can refer to the relative amount of polymer in the core, or the amount of pharmaceutically active agent in the shell. In some embodiments, the core contains no more than 25%, 20%, 15%, 10%, 7.5%, 5.0%, 2.5%, 1% or 0.5% (w/w) of shell polymer. In some embodiments, the shell contains no more than 25%, 20%, 15%, 10%, 7.5%, 5.0%, 2.5%, 1% or 0.5% (w/w) of pharmaceutically active agent.

The combination of high encapsulation efficiency and uniform diameters provides controlled release of the pharmaceutically active agent from the core/shell nanomaterial. Previous attempts to prepare core/shell nanomaterials for drug delivery often encountered burst release of the active agent upon exposure to a biological system. The core/shell nanomaterials can be characterized by the absence of “burst” release upon initial exposure to a biological system. The in vivo release profile can be estimated by measuring release in a system intended to mimic in vivo conditions. For instance, when the core/shell nanomaterials are immersed in 0.01 M PBS (phosphate buffered saline) (pH 7.4) at 37° C., no more than 0.1%, 0.5%, 1%, 2%, 3%, 4%, 5%, 6%, 7%, 8%, 9% or 10% of the pharmaceutically active agent is released within 24 hours. The rate of release can be controlled through proper selection of the biodegradable, biocompatible polymer as well as the relative thickness of the shell material. In some embodiments, at least 50%, 60%, 65%, 70%, 75%, 80%, 85%, 90% or 95% of the pharmaceutically acceptable agent is released within a period of 7, 10, 14, 21, 28, 35, 42, 49, 56, 63, 70, 77, 84, 91 or 98 days.

In some embodiments, the pharmaceutically active agent (sometimes designated “drug”) can be a biological macromolecule, small molecule drug, or a mixture of two or more biological macromolecules and/or small molecule drugs. Unless explicitly specified to the contrary, the term “pharmaceutically active agent” embraces both single agents and mixtures of multiple agents. Typically, small molecule drugs are characterized by a molecular weight no greater than 1,000 Daltons. Exemplary classes of pharmaceutically active agents include cancer chemotherapeutics, immunosuppressants, antibiotics, analgesics and anesthetics, and the like. In certain embodiments, the pharmaceutically active agent can include one or more cancer chemotherapeutics, such as an alkylating agent, an antimetabolite, an anti-microtubule agent, a tyrosine kinase inhibitor, a topoisomerase inhibitor, a CSF-1R inhibitor, or a cytotoxic antibiotic.

In some instance, the chemotherapeutic agent can include alkylating agents, for instance, uracil mustard, chlormethine, cyclophosphamide, ifosfamide, melphalan, chlorambucil, pipobroman, triethylenemelamine, triethylenethiophosphoramine, temozolomide, thiotepa, busulfan, carmustine, lomustine, streptozocin, and dacarbazine, antimetabolites, for instance methotrexate, 5-fluorouracil, floxuridine, cytarabine, 6-mercaptopurine, 6-thioguanine, fludarabine phosphate, pentostatin, pemetrexed, raltitrexed, cladribine, clofarabine, mercaptopurine, capecitabine, nelarabine, azacitidine and gemcitabine, EGF Pathway Inhibitors such as sunitinib, tyrphostin 46, imatinib, EKB-569, sorafenib, erlotinib, pazopanib, gefitinib, and lapatinib, vinca alkaloids such as vinblastine, vincristine, vindesine, and vinorelbine, cyclin dependent kinase inhibitors such as olomoucine, purvalanol B, roascovitine, indirubin, kenpaullone, purvalanol A and indirubin-3′-monooxime, proteasome inhibitors such as aclacinomycin A, gliotoxin, bortezomib, carfilzomib and ixazomib, platinum-based agents including carboplatin, cisplatin, and oxaliplatin, mTor inhibitors such as rapamycin, ridaforolimus, temsirolimus and SDZ-RAD, anthracyclines such as daunorubicin, doxorubicin, epirubicin, idarubicin, mitoxantrone, and valrubicin, topoisomerase inhibitor such as topotecan, irinotecan, etoposide, teniposide, and camptothecin, taxanes such as paclitaxel, docetaxel, larotaxel, and cabazitaxel, epothilones such as ixabepilone, epothilone B, epothilone D, BMS310705, dehydelone, and ZK-Epothilone (ZK-EPO), antibiotics such as actinomycin, plicamycin, daptomycin bleomycin, hydroxyurea, and mitomycin, immunomodulators including lenalidomide and thalidomide, HSP90 inhibitors like geldanamycin, anti-androgens including nilutamide and bicalutamide, antiestrogens such as tamoxifen, toremifene, letrozole, testolactone, anastrozole, bicalutamide, exemestane, flutamide, fulvestrant, and raloxifene, CSF-1R inhibitors such as GW2580. In other cases, the pharmaceutically active agent can include a biological macromolecule, for instance a therapeutic protein, monoclonal antibodies, vaccine, RNA or DNA.

The shell material can include one or more biocompatible, biodegradable polymers. As used herein, a biocompatible, biodegradable polymer is a polymer which can be broken down in vivo to monomer and/or oligomer fragments, wherein the monomeric or oligomeric fragments do not provoke an immune response, are not toxic, and can be easily excreted. Exemplary biocompatible, biodegradable polymers include poly(ethylene glycols) polyesters, mixed polyesters, for instance PLGA, polyanhydrides, mixed polyanhydrides, poly(ester)anhydrides, polysaccharides, polyphosphazenes, and copolymers. In some embodiments, the biocompatible, biodegradable polymer is sufficiently hydrophobic to control the release of the pharmaceutically active agent. The shell polymer can have a contact angle greater than about 90°, greater than about 100°, greater than about 110°, greater than about 120° greater than about 130°, greater than about 140° greater than about 150°, or greater than about 160°. In some embodiments, the shell polymer can have a contact angle between about 90-150°, between about 100-150°, between about 110-150°, between about 120-150°, or between about 125-150°. Generally, the core polymer, when present, can be hydrophilic, and can be water soluble such that it degrades/dissolves within 3 hours, within 2 hours, within 1 hour or with 30 minutes of being immersed in water.

In certain embodiments the biocompatible, biodegradable polymer can include one or more of poly(lactic-co-glycolic) acid (“PLGA”), polycaprolactone, polylactide, polyglycolide, polyhydroxybutyric acid, poly(sebacic acid), poly[1,6-bis(p-carboxyphenoxy)hexane], and mixtures thereof.

In certain cases, polycaprolactone can be used in combination with other polymeric systems. Suitable other systems include poly(ethylene glycols) (“PEG”), and PEG copolymers. Exemplary copolymers include polycaprolactone-poly(ethylene glycol), which may further be appended with a functional group such as an amino, thiol, carboxylate and the like. Such functional groups can be used to append biomarkers, dyes, and targeting factors to the encapsulated composition. An especially preferred system include PCL/PCL-PEG-NH₂.

The core/shell nanomaterials may be prepared using electrospinning and electrospraying processes. The core/shell nanofibers can be prepared using an electrospinning process, whereas nanocrystalline drugs can be prepared using an electrospraying process, and then further encapsulated with an appropriate polymer. In other embodiments, nano-encapsulated compositions can be directly prepared using a voltage-switched electrospinning process.

Generally, to prepare core/shell nanofibers, a pharmaceutically active agent is dissolved in a first solvent, and a biodegradable, biocompatible polymer is dissolved in a second solvent. The first and second solvents should be capable of dissolving the pharmaceutically active agent and biodegradable, biocompatible polymer, respectively. In comparison with previous processes, the solvent systems can be either miscible or immiscible with each other. The solvent systems can include other excipients, for instance stabilizers, surfactants, antioxidants, and the like. In some embodiments, the first solvent will not contain any of the biocompatible, biodegradable polymer, and the second solvent will not contain any of the pharmaceutically active agent.

Suitable solvents include aprotic solvents like dimethylsulfoxide (DMSO), halogenated hydrocarbons like chloroform and methylene chloride, ethers like tetrahydrofuran (THF) and diethylether, carbonyl- or nitrile-containing compounds like dimethylformamide (DMF), acetone, acetonitrile, ethyl acetate, and the like. Suitable solvents can also include protic solvents such as water, organic acids like formic acid, acetic acid, propionic acid, trichloroacetic acid, chloroacetic acid, trifluoroacetic acid and the like, or alcohols like methanol, ethanol, ethylene glycol, glycerol, isopropanol, and n-propanol. In some embodiments, either the first or second solvent can be a mixture of two or more solvents. In some embodiments, the solvent can be a mixture of at least one organic acid and at least one apolar solvent. The ratio (v/v) of organic acid to apolar solvent can be from 1:1 to 99:1, 2:1 to 99:1, 3:1 to 99:1, 4:1 to 99:1, 5:1 to 99:1, 7.5:1 to 99:1, 10:1 to 12.5:1, 15:1 to 99:1, or 20:1 to 99:1. In certain embodiments, the ratio (v/v) of organic acid to apolar solvent can be at least 85:15, 87.5:1, 90:10, 92.5:7.5, 95:5, 97.2:2.5, 98:2 or 99:1. Preferred apolar solvents for combination with the organic acid include halogenated hydrocarbons. Preferred organic acids include formic acid, acetic acid and mixtures thereof. When the organic acid is a mixture of formic acid and acetic acid, the ratio (v/v) can be from 75:25 to 25:75, 60:40 to 40:60, or 50:50. When the first or second solvent include an organic acid as described above, the other solvent will typically contain an aprotic solvent immiscible with the organic acid-containing system. Suitable such solvents include DMF, DMSO, methylene chloride, alkanes like cyclohexane and heptane, and aromatics such as toluene and xylene.

The pharmaceutically active agent can be dissolved in the first solvent, for instance at a concentration from about 1-100 mg/ml, about 5-100 mg/ml, about 10-100 mg/ml, about 25-100 mg/ml, or about 25-75 mg/ml. The biocompatible, biodegradable polymer can be dissolved in the second solvent at a concentration from about 1-500 mg/ml, 10-500 mg/ml, 25-500 mg/ml, 25-400 mg/ml, 25-300 mg/ml, 25-250 mg/ml, 50-250 mg/ml, 100-250 mg/ml, or 100-200 mg/ml.

Coaxial electrospinning can be conducted using concentric spinneret nozzles. The core solution may be spun from a needle having a gauge from 15-34, from 15-30, from 20-30, or from 25-30. In some embodiments, the core solution may be spun from a needle having a gauge of at least 10, at least 15, at least 20, at least 25, or at least 30. The needle may be placed concentrically into a shell nozzle having an inner diameter that is no more than 0.2 mm, 0.3 mm, 0.4 mm, 0.5 mm, 0.6 mm, 0.7 mm, 0.8 mm, 0.9 mm, 1.0 mm, 1.2 mm, 1.4 mm, 1.6 mm, 1.8 mm or 2.0 mm. In some embodiments, the shell nozzle can have an inner diameter that is at least about 120%, 140%, 160%, 180%, 200%, 250%, 300%, 400%, or 500% the outer diameter of the core needle. In some embodiments, the shell nozzle can have an inner diameter that is between about 120-500%, between about 150-400%, between about 150-300%, or between about 150-250% the outer diameter of the core needle.

The flow rate of the core solution through the spinneret can be at least 0.05 ml/hr, at least 0.10 ml/hr, at least 0.15 ml/hr, at least 0.20 ml/hr, at least 0.25 ml/hr, at least 0.30 ml/hr, at least 0.35 ml/hr, at least 0.40 ml/hr, at least 0.45 ml/hr, or at least 0.50 ml/hr. The flow rate of the core solution through the spinneret can be between 0.05 ml/hr and 0.50 ml/hr, between 0.05 ml/hr and 0.40 ml/hr, between 0.05 ml/hr and 0.30 ml/hr, between 0.10 ml/hr and 0.30 ml/hr, or between 0.20 ml/hr and 0.30 ml/hr.

The flow rate of the shell solution through the spinneret can be at least 0.10 ml/hr, at least 0.20 ml/hr, at least 0.30 ml/hr, at least 0.40 ml/hr, at least 0.50 ml/hr, at least 0.60 ml/hr, at least 0.70 ml/hr, at least 0.80 ml/hr, at least 1.0 ml/hr, at least 1.25 ml/hr, or at least 1.50 ml/hr. The flow rate of the core solution through the spinneret can be between 0.10 ml/hr and 1.50 mg/hr, between 0.10 ml/hr and 1.0 ml/hr, between 0.20 ml/hr and 1.0 ml/hr, between 0.10 ml/hr and 0.50 ml/hr, or between 0.25 ml/hr and 0.75 ml/hr.

The applied voltage for the electrospinning can be between 1-100 KV, between 10-100 KV, between 10-75 KV, between 10-50 KV, between 10-40 KV, between 15-40 KV, between 15-30 KV, or between 15-25 KV. The distance from tip to collector can be at least 50 mm, at least 75 mm, at least 100 mm, at least 125 mm, at least 150 mm, at least 175 mm, at least 200 mm, at least 250 mm, or at least 300 mm. In some embodiments, the distance from tip to collector can be from 50-300 mm, from 75-250 mm, from 100-250 mm, or from 100-200 mm. After electrospinning, the collected fibers and encapsulated drugs can be immersed in water to remove residual pharmaceutically active agent from the surface of the fibers and capsules. Nanoparticles of polymer, e.g., PCL/PCL-NH₂-PEG, having a size range of 15-30 nm, 30-50 nm, or 5-100 nm can be prepared. In some cases, the electrospraying can be done with various polymers, for instance those having MW from 2,000-14,000 g/mole.

Nanocrystalline drugs can be also obtained by an electrospraying process. Typically, the drug to be crystallized can be dissolved in one or more of the solvents described above for electrospinning. In certain embodiments, volatile solvents can be used. Exemplary volatile solvents include methanol, ethanol, dichloromethane, acetone, diethylether, ethyl acetate and the like. In certain embodiments, the solvent can include an organic acid like formic acid or acetic acid to assist in the dissolution and stability of the compound. Organic acids can be added in an amount of from 0.01-1.0% (v/v), 0.05-1.0% (v/v), 0.05-0.5% (v/v), or 0.05-0.25% (v/v). The flow rate of the drug solution through the spinneret can be at least 0.25 ml/hr, at least 0.50 ml/hr, at least 0.75 ml/hr, at least 1.0 ml/hr, at least 2.0 ml/hr, at least 3.0 ml/hr, at least 4.0 ml/hr, at least 5.0 ml/hr, at least 6.0 ml/hr, at least 7.0 ml/hr, at least 8.0 ml/hr at least 9.0 ml/hr, or at least 10.0 ml/hr. The flow rate of the drug solution through the spinneret can be between 0.50 ml/hr and 10.0 ml/hr, between 2.0 ml/hr and 10.0 ml/hr, between 5.0 ml/hr and 10.0 ml/hr, between 0.50 ml/hr and 5.0 ml/hr, between 1.0 ml/hr and 5.0 ml/hr, or between 2.50 ml/hr and 5.0 ml/hr. The applied voltage for the electrospraying can be between 1-100 KV, between 1-75 KV, between 5-75 KV, between 5-50 KV, between 10-50 KV, between 10-25 KV, or between 25-50 KV. The distance from tip to collector can be at least 50 mm, at least 75 mm, at least 100 mm, at least 125 mm, at least 150 mm, at least 175 mm, at least 200 mm, at least 250 mm, or at least 300 mm. In some embodiments, the distance from tip to collector can be from 50-300 mm, from 100-250 mm, from 150-250 mm or from 175-225 mm. Nanocrystals can be collected on a plate, for instance a glass collector plate.

The particle size of the obtained nanocrystalline material can be further reduced using physical agitation, for instance, using sonication. One or more immiscible solvents can be added, and the particle size of the nanocrystals further reduced by sonication. The identity of the immiscible solvent will depend on the nature of the drug crystals. The skilled person can easily determine suitable immiscible solvents for any nanocrystalline drug. For the purpose of sonication, an immiscible solvent is one in which the solubility of the drug (at 23° C.) is less than 100 μg/ml, less than 50 μg/ml, less than 25 μg/ml, less than 10 μg/ml, less than 5 μg/ml, or less than 1 μg/ml. In some embodiments, the sonication can be conducted at 10-90%, 20-80%, 20-70%, 20-60%, 30-60%, or 30-50% intensity. The relative intensity is with respect to the full power capacity of the sonicator (750 watt). The sonication may be conducted from 0.5-10 hours, from 1-10 hours, from 1-8 hours, from 1-5 hours, from 1-3 hours.

In certain embodiments, nanocrystalline material can be collected from the electrospraying process in a collector which also serves as the vessel for sonication. Drug nanocrystals prepared by this method can have a particle size of less than 250 nm, less than 200 nm, less than 150 nm, less than 100 nm, less than 75 nm, less than 50 nm, less than 25 nm, less than 10 nm, or less than 5 nm.

The nanocrystalline material can be used encapsulated in a polymer using a variety of techniques. In certain embodiments, a phase inversion encapsulation process can be employed. The biodegradable, biocompatible polymer may be added directly to the nanocrystalline drug/immiscible solvent mixture, or the biodegradable, biocompatible polymer may be dissolved in a second solvent, optionally with one or more surfactants, and then combined with the nanocrystalline drug/immiscible solvent mixture. In either case, the components can be then agitated, for instance by stirring or sonication, while the immiscible solvent is evaporated. In cases where a second solvent is employed, removing the immiscible solvent generates the encapsulated nanocrystalline drug suspended in the second solvent. The second solvent may then be removed by evaporation or lyophilization.

In some embodiment, the nanocrystalline drug is paclitaxel or GW2580, either of which can be dissolved a combination of solvents of methanol and acetic acid. The solution can be electrosprayed using the NF500 state of the electrospinning machine, and the nanocrystalline drug collected in a glass collector. An immiscible solvent, e.g., dichloromethane (DCM) can be added and sonicated for several (1-3) hours at 30-50% intensity to give drug nanocrystals having less than 50 nm average size distribution. The polymeric components may then be added (e.g. PCL/PCL-PEG-NH₂) to the DCM/nanocrystalline material give an organic phase for an emulsion encapsulation process. The organic phase can be added, for instance by dropwise addition via syringe pump, to an inorganic phase. Exemplary inorganic phases include 1% PVA (Polyvinyl Alcohol) in deionized water, which may contain one or more surfactants, including polysorbate 80. In some cases, the mixture can be sonicated during addition or after addition is complete. The mixture can then be stirred while allowing the organic phase solvent to evaporate. The temperature may be maintained above the boiling point of the organic phase solvent but below the boiling point of the inorganic phase solvent. After the organic phase is removed, the suspended nanoparticles may be collected, for instance by centrifugation or filtration, and washed with water to remove any residual inorganic phase. The nanoparticles may be freeze dried to further remove residual water.

Nanoencapsulated drugs can also be directly obtained through an electrospraying process. Typically, the drug and biocompatible, biodegradable polymer are combined in one or more of the solvents described above for electrospinning. In certain embodiments, volatile solvents can be used. Exemplary volatile solvents include methanol, ethanol, dichloromethane, acetone, diethylether, ethyl acetate and the like. In certain embodiments, the solvent can include an organic acid like formic acid or acetic acid to assist in the dissolution and stability of the compound. Organic acids can be added in an amount of from 0.01-1.0% (v/v), 0.05-1.0% (v/v), 0.05-0.5% (v/v), or 0.05-0.25% (v/v). In some embodiments, the flow rate of the drug/polymer solution through the spinneret can be at least 0.05 ml/hr, at least 0.10 ml/hr, at least 0.15 ml/hr, at least 0.20 ml/hr, at least 0.25 ml/hr, at least 0.50 ml/hr, at least 0.75 ml/hr, or at least 1.0 ml/hr. In certain embodiments, the flow rate of the drug/polymer solution through the spinneret can be no more than 0.05 ml/hr, no more than 0.10 ml/hr, no more than 0.15 ml/hr, no more than 0.20 ml/hr, no more than 0.25 ml/hr, no more than 0.50 ml/hr, no more than 0.75 ml/hr, or no more than 1.0 ml/hr. The flow rate of the drug/polymer solution through the spinneret can be between 0.05 ml/hr and 1.0 ml/hr, between 0.10 ml/hr and 1.0 ml/hr, between 0.20 ml/hr and 1.0 ml/hr, between 0.20 ml/hr and 0.5 ml/hr, between 0.5 ml/hr and 1.0 ml/hr, or between 0.75 ml/hr and 1.0 ml/hr. The applied voltage for the electrospraying can be between 0.1-50 KV, between 1-50 KV, between 5-50 KV, between 5-25 KV, or between 10-25 KV. The distance from tip to collector can be at least 50 mm, at least 75 mm, at least 100 mm, at least 125 mm, at least 150 mm, at least 175 mm, at least 200 mm, at least 250 mm, or at least 300 mm. In some embodiments, the distance from tip to collector can be from 50-300 mm, from 100-250 mm, or from 150-250 mm. Encapsulated drugs may be collected on a plate, for instance a glass collector plate.

The nanoencapsulated drugs may be combined with one or more targeting vectors or biomarkers/Fluorescent dye/IR Dye. For instance, when the polymer includes PCL-PEG-NH₂, (2000 MW) the nanoparticles can be coupled to IRdye650 for the proposed targeting therapies. For example to tag with IR Dye 650 NHS, the nanoparticles are dispersed in an PBS/MES Buffer solution (deprotonate the amine group/activated) that is added with appropriate amount of a nanoparticles that is stirred overnight at room temperature to complete the coupling reaction. Stock solutions of concentration 1 mg/1 mL were made of both the PCL-PEG-NH₂ and the IR dye. The functionalized nanoparticles are recovered using a high-speed centrifuge, and vacuum drying and or through Size dependent dialysis tubes, benzoylated (2000MWCO) and then rinsed thoroughly using deionized water to remove any impurities for the following cell studies.

Similarly, the schematic for the synthesis of PCL-PEG-NH₂ nanoparticles coupled with NIR emission dye that has a maximum emission wavelength of 650 nm is shown in FIG. 24.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Core/Shell Nanofibers

Polycaprolactone (PCL) of average molecular weight 80,000 g/mol, polyvinyl alcohol (PVA) of average molecular weight 89-98,000 g/mol was purchased from Sigma Aldrich. Anticancer drug 5-Fluorouracil (FU) of 98% purity was purchased from AK Scientific. All other chemicals were of reagent grade and purchased from Sigma-Aldrich unless otherwise specified. NF-500 Electrospinning Unit (MECC, Japan) was utilized for electrospinning process. Ultra coaxial spinneret, a special concentric nozzle was used for developing coaxial stream core and sheath. 27G needle was used as the core nozzle, which was placed concentrically into shell nozzle (0.8 mm). Solutions was pumped to the spinneret using two separate precision syringe pumps and 10 ml NORM-JECT® latex free syringes. A separately attached dehumidifier was attached to control system humidity.

Six sets off fluorouracil (FU) loaded PCL nanofibers (NFs) were fabricated using different solvent systems and electrospinning parameters. Solvent systems and electrospinning parameters for all sets of NFs are summarized in Table 1. For NF set A, 140 mg/ml of PCL was dissolved in DMF at 110° C. on a magnetic stirrer. For all other NFs, a shell solution of 140 mg/ml PCL in a mixture of solvents of 47.5% FA, 47.5% AA, 5% TCM (trichloromethane) was used. In case of core solution for NFs sets B and C, 50 mg/ml of PVA was added in addition to 50 mg/ml FU. Neat PCL was manufactured as a control sample using the sheath solution used to synthesize NFs set A.

| | | | | | | | | Tip to |
|---|---|---|---|---|---|---|---|---|
| | Core solution | | | Sheath solution | | | Applied | collector |
| | | | Flow rate | | Solute | Flow rate | Voltage | distance |
| # | Sol. | Solute (mg/ml) | (ml/hr) | Solvent | (mg/ml) | (ml/hr) | (KV) | (mm) |
| | N/A | N/A | N/A | DMF | 140 mg/ml | 1 | 21 | 185 |
| | | | | | PCL | | | |
| A1 | DMF | 50 mg/ml FU | 0.2 | DMF | 140 mg/ml | 1 | 21 | 185 |
| | | | | | PCL | | | |
| B2 | FA | 50 mg/ml FU, | 0.4 | 47.5% FA, 47.5% | 140 mg/ml | 1 | 22 | 135 |
| | | 50 mg/ml PVA | | AA, 5% TCM | PCL | | | |
| C2 | FA | 50 mg/ml FU, | 0.5 | 47.5% FA, 47.5% | 140 mg/ml | 1 | 23.2 | 110 |
| | | 50 mg/ml PVA | | AA, 5% TCM | PCL | | | |
| D3 | FA | 50 mg/ml FU | 0.2 | 47.5% FA, 47.5% | 140 mg/ml | 0.6 | 19.5 | 135 |
| | | | | AA, 5% TCM | PCL | | | |
| E3 | FA | 50 mg/ml FU | 0.2 | 47.5% FA, 47.5% | 140 mg/ml | 0.4 | 21 | 135 |
| | | | | AA, 5% TCM | PCL | | | |
| F3 | FA | 50 mg/ml FU | 0.2 | 47.5% FA, 47.5% | 140 mg/ml | 0.3 | 19 | 135 |
| | | | | AA, 5% TCM | PCL | | | |
| G3 | FA | 5 mg/ml | 0.1 | 47.5% FA, 47.5% | 140 mg/ml | 0.6 | 19.5 | 135 |
| | | Paclitaxel | | AA, 5% TFE | PCL | | | |

Key: dimethylformamide (DMF), Acetic Acid (AA), formic acid (FA), polyvinyl alcohol (PVA) trichloromethane (TCM), trifluoroethanol (TFE).

Morphology of coaxial nanofibers is influenced by solution properties, as well as by drug/polymer/solvent interactions. Conductivity difference, viscosity difference, surface tension and volatility of solvents are some of the important physical properties which affects uniformity of core-sheath nanofibers. Viscosity and surface tension difference between solutions dictates Rayleigh instability or jet breakup which primarily determines the presence (or absence) of beads.

| | Core solution | Voltage | | Flow ratio | Encapsulation | Avg dia |
|---|---|---|---|---|---|---|
| # | characteristics | (KV/cm) | Morphology | (Shell/core) | efficiency (%) | (nm) |
| A1 | High conductive and low | 1.135 | Large beads | 5 | 70.48 | 120 |
| | volatility | | | | | |
| B2 | Low conductive, | 1.629 | Outside | 2.5 | 62.96 | 238 |
| | high volatility, | | drug | | | |
| | contains PVA | | particles | | | |
| C2 | Low conductive, | 2.109 | Outside | 2 | 52.71 | 170 |
| | high volatility, | | drug | | | |
| | contains PVA | | particles | | | |
| D3 | Low conductive, high | 1.444 | Uniform | 3 | 73.62 | 170 |
| | volatility | | | | | |
| E3 | Low conductive, high | 1.555 | Uniform | 2 | 72.98 | 200 |
| | volatility | | | | | |
| F3 | Low conductive, high | 1.407 | Uniform | 1.5 | 77.5 | 220 |
| | volatility | | | | | |
| G3 | Low conductive, high | 1.444 | Uniform | 6 | 76.11 | 46.8 |
| | volatility | | | | | |

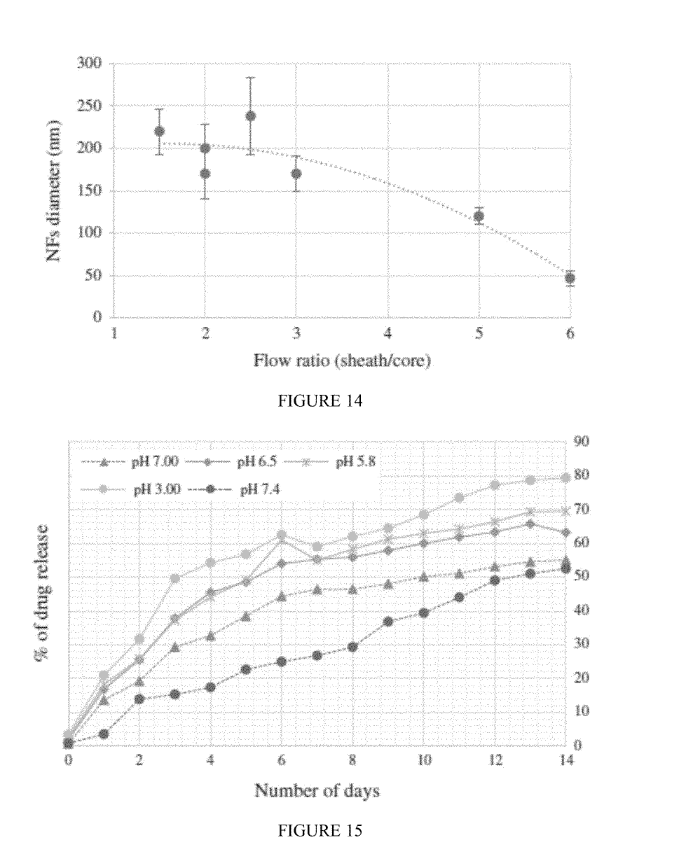

Category 2 NFs (B2, C2) exhibited accumulation of drug particles outside of fibrous structure. Both of these experienced higher electric potential (1.76 KV/cm and 2.109 KV/cm) in comparison to other NFs set when voltage is considered as a function of tip to collector distance. Such high electric potential causes free charges or ion to migrate very rapidly from core fluids and core-sheath interface to the free surface of the shell. Such rapid migration of charges results in penetration of shell layer since FU solution is polar in nature and can carry ion. When applied voltage is low, charge accumulation occurs primarily on the shell solution and the compound solution droplet at the tip of the spinneret can be expected to remain stable. The electrospinning process can then proceed via charge repulsion at the surface of the shell solution. Entrainment of the core solution can subsequently occur through viscous dragging and contact friction at the interface (of both solutions). Core solution for NFs set B and C also contains PVA in addition to drug which increases its surface tension. FU and PVA are not mutually miscible. PVA possesses higher electrospinability while drug solution alone tends to spray. FIG. 13 demonstrates the correlation between drug encapsulation efficiency and effective applied voltage. FIG. 14 demonstrates the correlation between sheath/core flow ratio and diameter.

Formic acid, acetic acid and TFE can individually be used, or used in combination, as a solvent for PCL which results in different NF morphology due to the variation of conductivity, viscosity of resulting solution and volatility difference.

All NFs were collected on wax paper using flat plate collector at traverse speed of 10 mm/s of spinneret and 150 mm traverse distance to produce long and thin nanofibers. Nanofibers were taken out of the wax paper and immersed in 50 ml deionized water at 37° C. for two hours to let the nanofibers release any drug attached to its surface. It was then dried in vacuum oven at 25° C. for 24 hours. FIG. 11 depicts an energy dispersive x-ray spectrum of NFs B2 and C2.

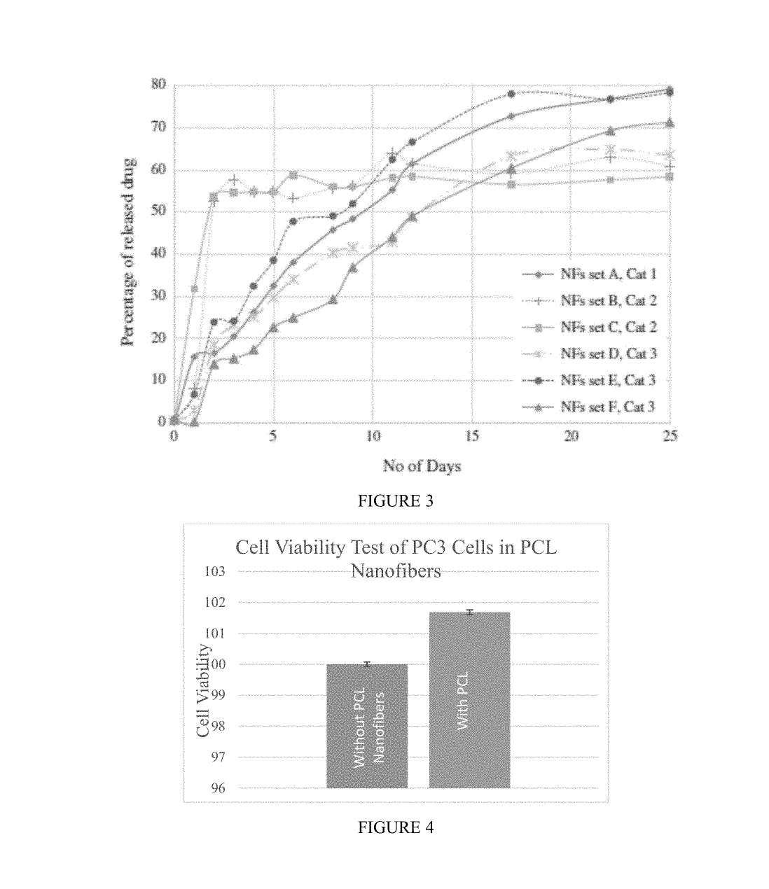

In-vitro drug release of all sets of NFs for 25 days in PBS media is shown in FIG. 3. Category 1 and 3 NFs (D3, E3, F3, and G3) exhibited steady and consistent release pattern throughout the entire testing period. NFs set A, category 1 showed little bit of high release on the first day while NFs set F, category 3 didn't release significant FU within first day. After first day of drug release NFs set A, F exhibited very similar release pattern though percentage of released drug from NFs set A were approximately 10% higher than NFs set F. On the other hand category 2, NFs set B and C exhibited very high drug release percentage during the initial time period. Both of them released 52-53% of encapsulated within three days of release test while NFs set A, D, E and F released only 13-23% drug. Drug release rate of NFs set B and C were significantly reduced after 3^{rd} day while other sets of NFs showed consistent release pattern. Drug encapsulation efficiency of NFs set A, B, C, D, E and F were determined 70.48%, 62.96%, 52.71%, 73.62%, 72.98%, 77.5% and 76.11% respectively.

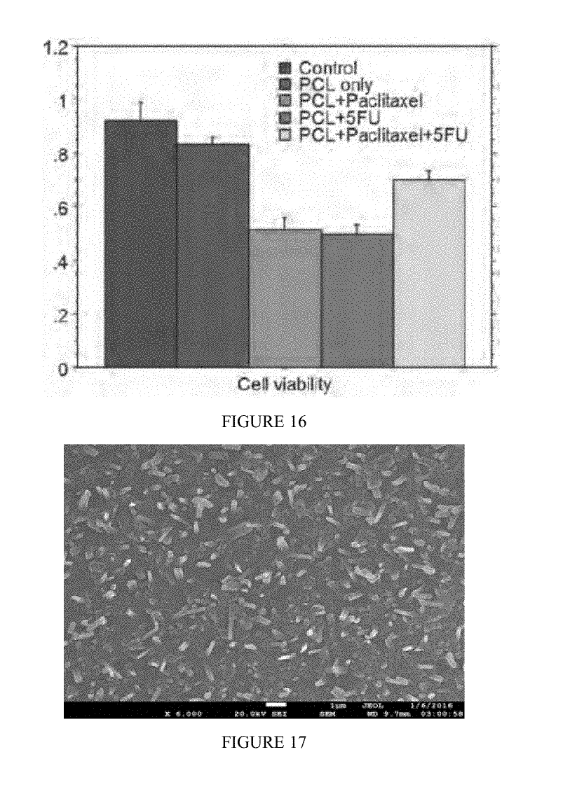

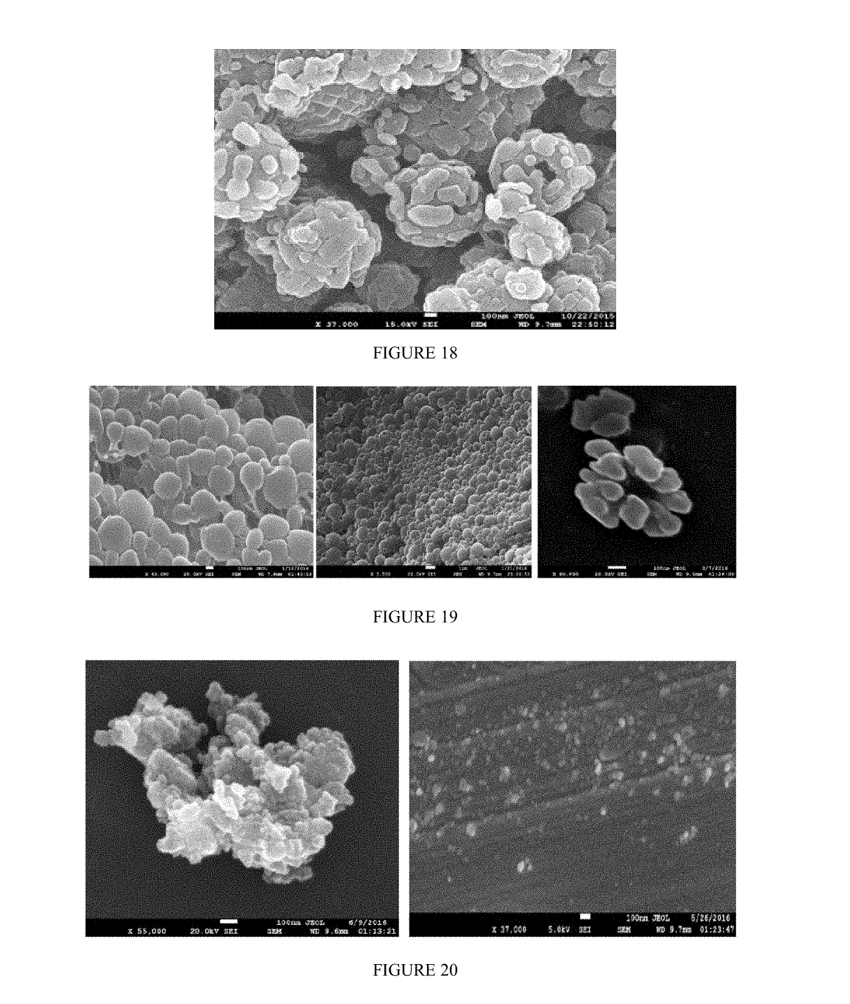

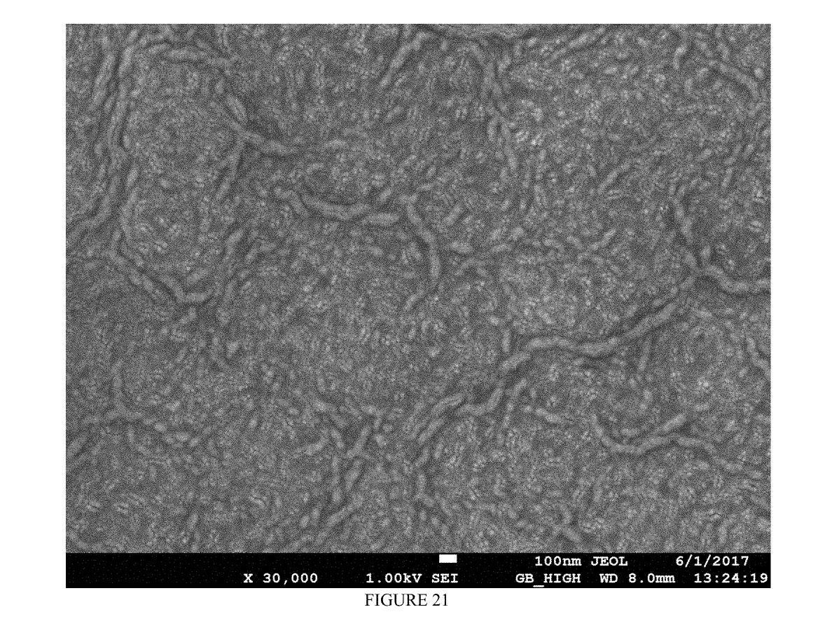

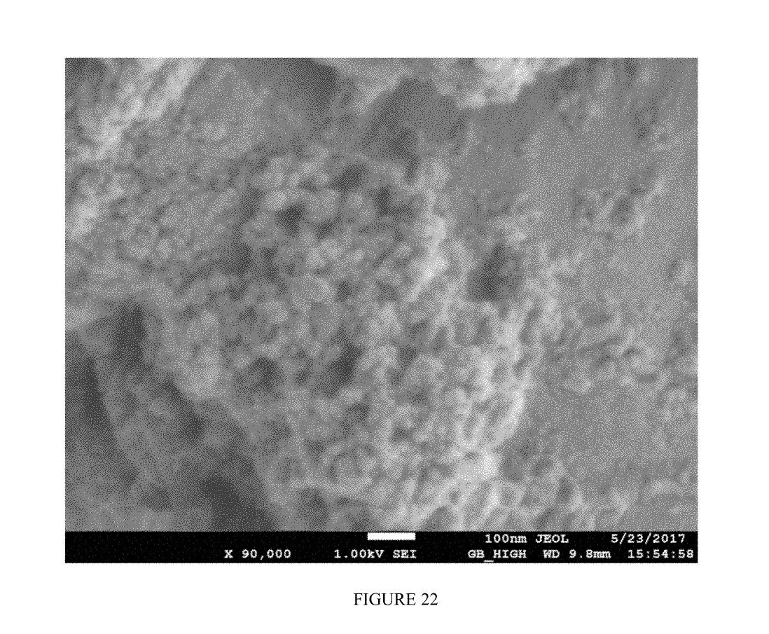

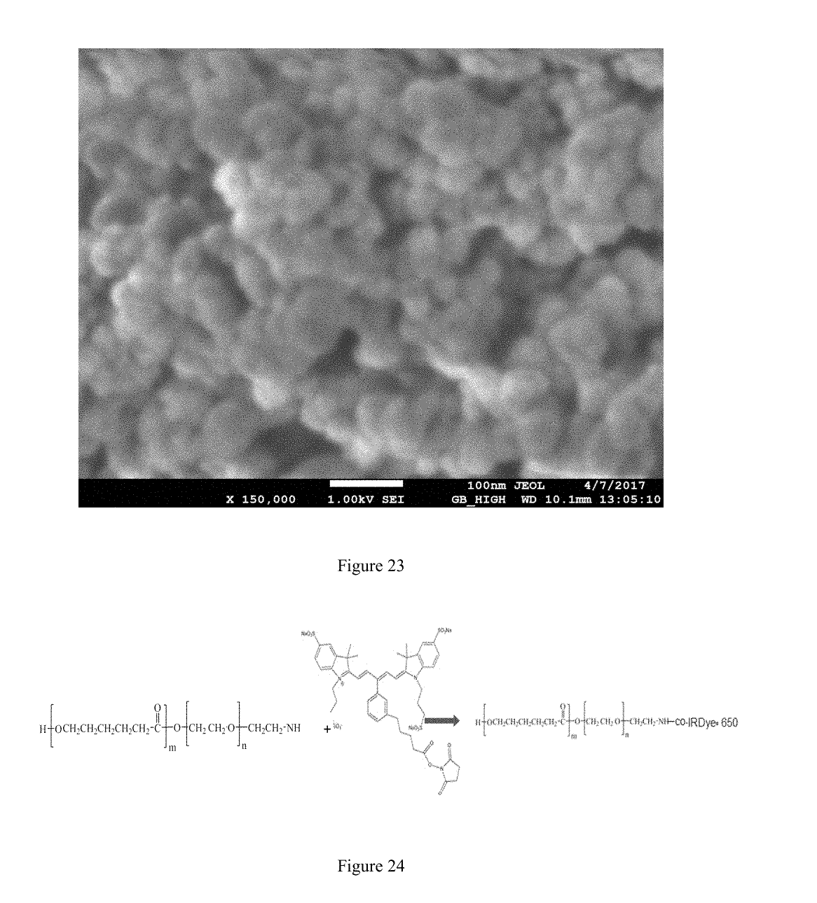

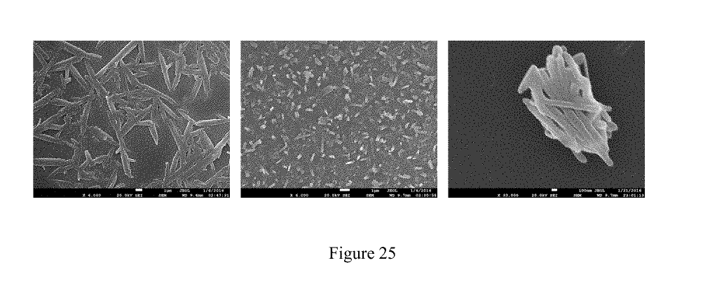

In vitro drug release test of FU loaded PCL nanofibers in PBS media at different pH is shown in FIG. 16. Four different pH level (7.0, 6.5, 5.8 and 3.0) of PBS media were used. Four sets of FU loaded PCL nanofiber samples were tested at each pH level. The results of the release tests are shown in FIG. 10. As demonstrated in the FIG. 15, effect of pH was prevalent from the starting of drug release period. It is observed that, % of drug release shows an increasing trend with decreasing pH level of the PBS solution. Lower pH level increase the degradation rate of nanofibers which expedites release rate. NFs released least amount of drug in 7.4 pH and drug release rate increase significantly while pH was reduced from 7.4 to 7.00 pH and 6.50 pH. NFs exhibited almost similar release pattern at 6.5 and 5.8 pH. It is also observed that drug release rate increased significantly with slight change of pH from 7.4. But when the pH of PBS is already low (such as 5.8), it requires a significant change of pH to increase a small amount of drug release rate. For example in our experiment on 4^{th} day of drug release test, only 0.4 decrease of pH from 7.4 to 7.0 increased 15.3% drug release while decrease of 2.8 pH from 5.8 to 3.00 increased only 10.2% of drug release rate.

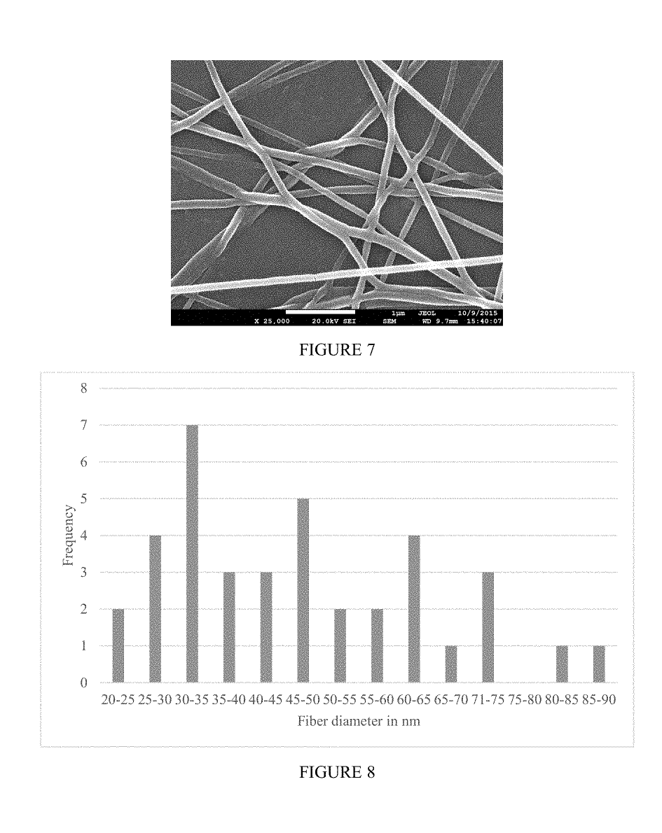

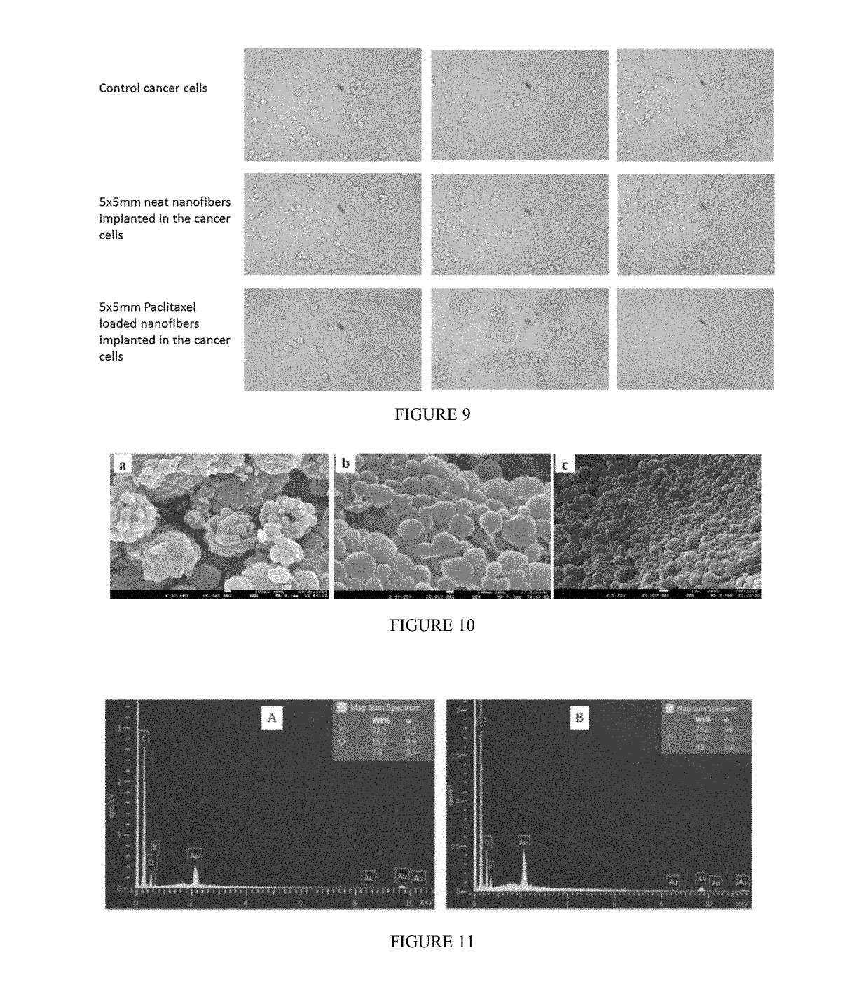

SEM image of Paclitaxel loaded PCL nanofibers were shown in FIG. 7. SEM images of Paclitaxel loaded PCL nanofiber exhibited thin nanofibers with rough surface properties. Diameter of nanofibers were randomly distributed from 22-90 nm at average diameter of 46.8 nm and standard deviation of 17.2 nm. FIG. 9 depicts diameter distribution of the nanofibers.

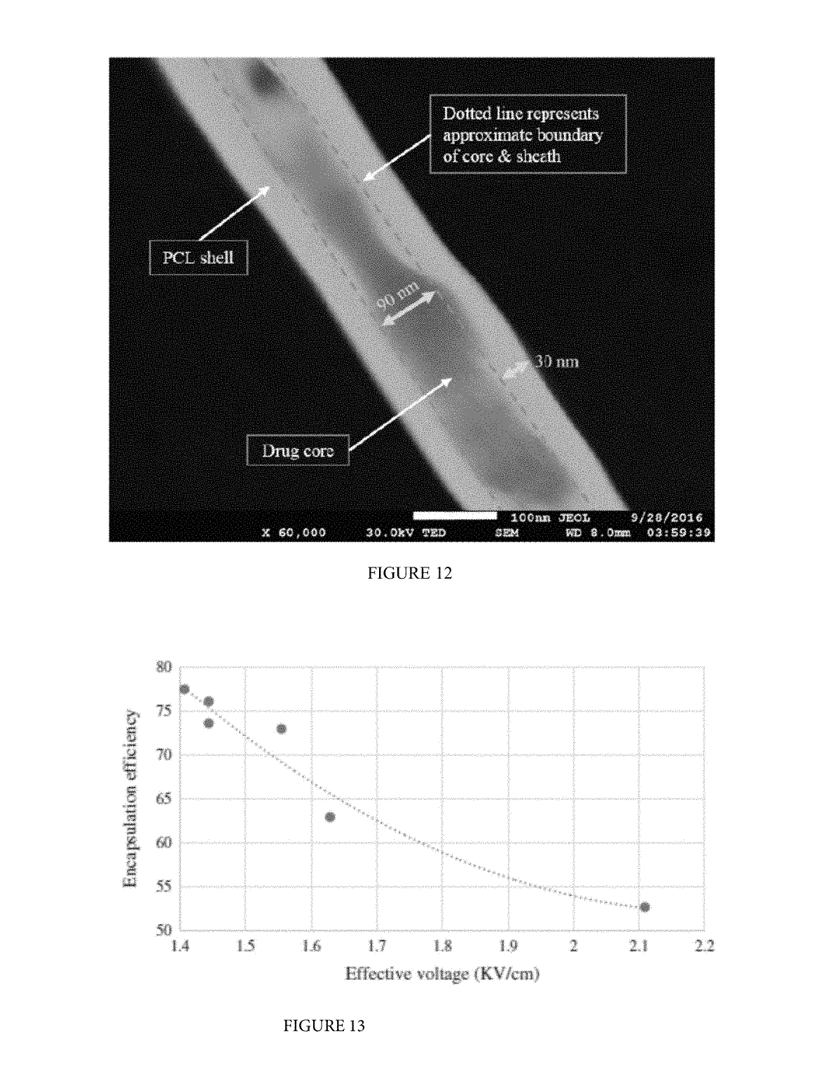

In another example, NaCl was added to polymer sheath and another cancer drug cisplatin (contains platinum) was added along with FU in the core solution. All other solution processing and electrospinning parameters were kept as same as NFs set F. Nanofibers were electrospun directly on TEM grid and dried in vacuum chamber for 24 hours. TEM image as shown in FIG. 12 exhibited drug core of app 80-90 nm and polymer sheath of 30-40 nm thickness.

In Vitro Drug Release Test:

Six random samples of size approximately 20×20 mm were taken from each set of NFs. Weight of the samples of corresponding NF set were noted down. Samples were put into 14 ml of 0.01M Phosphate Buffer Saline (PBS) in centrifuge tubes and preserved in a shaking incubator at 37° C. to allow the NFs to release drug in PBS. 50 μL of PBS solution were collected from each centrifuge tubes to UV transparent 96 well plate at 24 hours interval to measure the amount of released drug from nanofibers within that time. A vortex mixer was used to make the solution homogenized before taking samples. Samples absorbance were measured using UV-Vis spectrometer at 265 nm. Concentration of FU was calibrated using 38 solutions of Hi at different known concentration. To determine total encapsulated drug within the fiber membrane 15 mg of NF's from each set of NF's were dissolved into 14 ml dichloromethane and their absorbance were measured at 265 nm. Background absorbance of dichloromethane: was deducted to get absorbance of 5-FU which were compared with standard FU solution. Drug encapsulation efficiency was determined by comparing the encapsulated drug with the feed rate of drug in fiber manufacturing process. In order to investigate the effect of differential pH level on the drug release characteristics, release tests were performed at different pH level Results are depicted in FIGS. 3 and 15.

In Vitro Cytotoxicity Test:

Human prostatic cancer cell PC3 were used to test the biocompatibility and cytotoxicity of FU loaded PCL nanofibers. Human prostatic cancer cell derived from metastatic site (bone) was purchased from ATCC, USA. 57 ml of fetal bovine serum (USA origin purchased from Sigma Aldrich), 7 ml of Penicillin-Streptomycin (Life Technologies) and 6 ml of HEPES (Life Technologies) was added to 500 ml RPMI-1640 medium (Fisher Scientific). This media is called R+ media. 96 well plate were used to grow PC3 cells (sterile and flat bottom type purchased from fisher scientific). 100 μL of PC3 cells at 8×10⁴ cells/ml were added on each wells of 96 well plate for all tests. PC3 cells were incubated at 37° C. in a humidified 5% CO₂ atmosphere for 24 hours before performing any test.

Cytotoxicity tests were performed for neat PCL nanofibers, pristine FU, NFs set A and D. Each tests were performed in individual well plates to avoid cross contamination. All nanofiber samples were sterilized using UV radiation for one hour before performing cytotoxicity test. Neat PCL nanofibers of 5×5 mm dimension were incorporated directly into PC3 cells to investigate biocompatibility of PCL NFs. Biocompatibility test were carried out using 6 replica of neat PCL NFs and 6 pristine PC3 cells as control sample. Cytotoxicity of pristine FU was performed using an aqueous solution of FU of concentration 0.33-5.6×10⁻⁶ μM at 1/3 dilution in each step. FU solutions were directly incorporated into cell culture media. Tests were performed in triplicates of each FU concentration.

The cytotoxicity of NFs set A and D was assessed. Both NF samples were immersed into 14 ml R+ media and incubated at 37° C. for an hour. This R+ media was later diluted by fresh R+ media at different concentration from 0.33-5.6×10⁻⁶ μM of FU. These FU loaded media was used for the cell culture of PC3 cells.

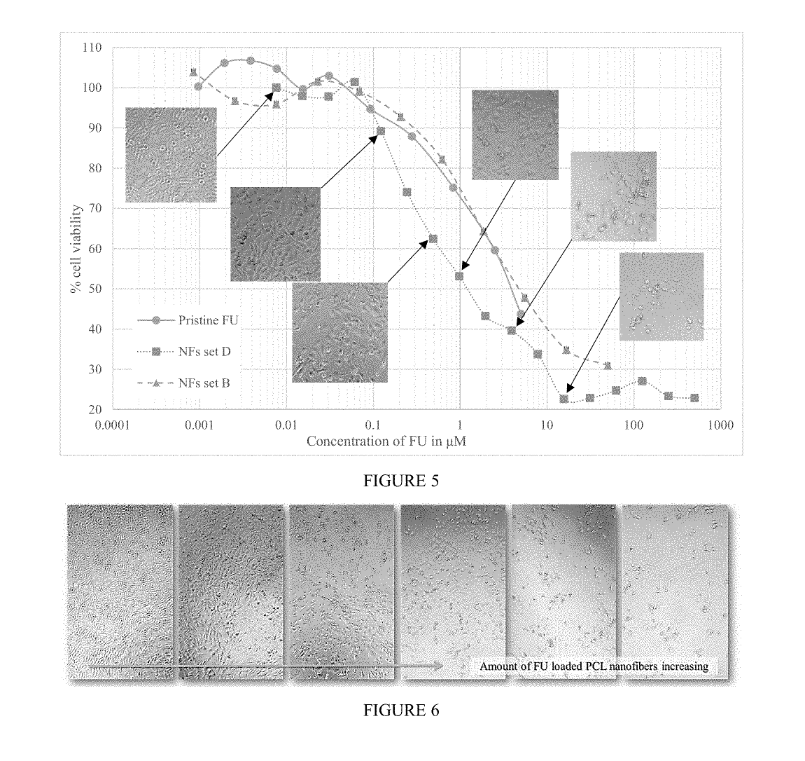

96 well plates were incubated at 37° C. in a humidified, 5% CO₂ atmosphere for 48 hours to give sufficient time for the fluorouracil to inhibit essential biosynthetic process of the PC3 cells and thus cause cell death. Antimetabolite drug takes 2-5 days to cause a cell to die. After 48 hours, well plates were taken out from incubator and 20 μL of MTS-based CellTiter solution was added in each well. MTS based cell titration is a convenient colorimetric method to determine the number of viable cell in cytotoxicity assay. Well plates were kept again in CO₂ incubator for 3 hours and then their absorbance were measured at 490 nm using UV-Vis spectrometer. Absorbance of cells with different concentration of drug was compared with the absorbance of PC3 cells with no drug to determine the cell viability. Percentage of cell viability was plotted against drug amount which indicates the killing curve of neat FU, NFs set A & D (FIG. 5).

Paclitaxel loaded nanofibers were tested in human breast cancer cells for 48 hours. Nanofiber membranes were directly incorporated into cell culture media to test its cytotoxicity. Cells were cultured with only cancer cells, cells with neat PCL and Paclitaxel loaded PCL nanofibers. Cell viability was evaluated at 24, 48 and 72 hour intervals using an optical microscope. Microscope images of cancer cells is shown in FIG. 9.

Paclitaxel loaded nanofibers were tested in human breast cancer cells for 72 hours. The drug paclitaxel was delivered in thin film-like forms as Paclitaxel-PCL. They were then cut into small 3 mm×3 mm square pieces that weighed 0.0576 mg containing 0.002592 mg of Paclitaxel for Paclitaxel-PCL. These small pieces were then stored in 100 mm petri dishes and disinfected inside the biosafety laminar flow hood with ultra violet (UV) light for 18 hours.

MDA-MB-231 breast cancer cells (4×10⁴ cells) per well were seeded in 24 wells plate. The cells were maintained in 500 μl DMEM-high glucose media with 10% FBS and incubated at 37° C. for 24 hours in normoxia chamber (21% O₂, 5% CO₂) to allow the cells to attach and get acclimatized to the growth conditions. After 24 hours, the media was aspirated gently and the cells were washed with 1×PBS. 500 μl DMEM-HG fresh media was added to each well gently along the walls. The pieces of NFs containing paclitaxel were added in the increments of one each starting from one piece per well to four pieces per well. Each treatment was performed in duplicates. The cells with the treatments were incubated for 24 hrs, 48 hrs and 72 hrs following which images were obtained at each time point using an inverted microscope. Cells were cultured with only cancer cells, cells with neat PCL and Paclitaxel loaded PCL nanofibers.

The drug Paclitaxel and 5-FU was delivered in thin film-like forms as Paclitaxel-PCL and Paclitaxel-5 FU-PCL and 5 FU-PCL. They were then cut into small 3 mm×3 mm square pieces that weighed 0.00065 g containing 0.00004 g of Paclitaxel for Paclitaxel-PCL and weighed 0.00067 g containing 0.0000388 g of 5-FU for 5 FU-PCL and weighed 0.00062 g containing 0.000031 Paclitaxel & 0.0000124 5-FU. These small pieces were then stored in 100 mm petri dishes and sterilized inside the biosafety laminar flow hood with ultra violet (UV) light for 18 hours.

The WST assay was used as a cytotoxicity assay for the Human Triple Negative Breast Cancer (TNBC) cell line, MDA-MB-231 cell line using Paclitaxel, 5-FU and combination of Paclitaxel-5FU. 10,000 MDA MB-231 cells were seeded into 96-well plates (costar 3596-Corning Incorporated) in 100 μL of high glucose concentration Dulbecco's Modified Eagle Medium (DMEM) supplemented with 10% fetal bovine serum, 4.0 Mm L-Glutamine and 4500 mg/L Glucose as provided by the manufacturer (Thermo Scientific). There were total five experimental conditions—i) control, ii) PCL fiber only, iii) PCL & Paclitaxel, iv) PCL & 5-FU, v) combination of PCL, Paclitaxel & 5-FU. A chipped piece of drug (as mentioned in the preparation of paclitaxel section above) from each formulation was added into the corresponding well. Each treatment was performed in triplicates for achieving statistical significance. The cells with the treatments were incubated at 37° C. in a normoxia (21% O₂) chamber for 24 hours. After 24 hours the chipped piece of drug was removed from each well. Then 10 μL of WST-1 reagent (ROCHE) was added to each well and incubated at 37° C. in a normoxia (21% O₂) chamber for 4 hours in dark. The remaining viable cells with WST dye uptake were determined by measuring the optical density at 490 nm in Perkin Elmer Wallac 1420 Victor Multilabel Counter.

Trypan Blue Exclusion Test of Cell Viability:

Trypan Blue dye exclusion test was used to discriminate between viable and non-viable cells. 100,000 Human Triple Negative Breast Cancer (TNBC) cell line, MDA-MB-231 were seeded into 24-well plate (costar 3527-Corning Incorporated) in the same media with the same conditions. After 24 hours, all cells (both floating and attached cells) were collected and the viability was determined under bright field microscope by adding trypan blue dye. Dye positive cells are considered dead cells.

In-vitro biocompatibility test of neat PCL NFs is shown in FIG. 4. Cell viability of PC3 cells in neat PCL was evaluated using MTS assay. Cell viability test showed slightly (about 2%) increased growth of PC3 cell in neat PCL NFs in comparison to PC3 cells without any NFs. This further demonstrates that PCL NFs itself not toxic to cells. Nanofibers possess three dimensional architecture which is more favorable to cell growth in comparison to flat bottom cell culture microplate.

In vitro cytotoxicity test of pristine FU, NFs set A and NFs set D is shown in FIG. 5. Microscopic images demonstrates PC3 cells at corresponding concentration after 48 hours of exposing NFs to the cell culture media. NFs set A exhibited similar cytotoxic effect to pristine FU while NFs set D demonstrated enhanced efficacy and killing rate. At 5 μM initial drug concentration in the media NFs set D exhibited 38% alive cells while in pristine FU 43% cells were alive and in NFs set A 47.8% cells were alive. With increased concentration of NFs (set D) we have observed the cell viability decreased close to 20%. Drug release from nanofiber matrix relies on diffusion phase from the polymeric matrix along with the biological degradation of the carrier. Diffusion is responsible for initial burst release and in later stage drug release mainly relies on degradation of polymeric shell. In comparison to polylactic acid (PLA), poly glycolic acid (PGA), and other aliphatic polymers, the degradation of PCL in a buffer solution is very slow because of its semi-crystalline nature. The degradation of implanted, sub-dermal, drug delivery devices produced from PCL (Mn 50,000) over a timescale of 52 months has been evaluated. Weight loss was not recorded until after approximately 2.5 years when the molecular weight (Mn) had fallen to 5000, reflecting the requirement for degraded chain fragments to be below a critical chain length for diffusion from the matrix.

Paclitaxel loaded PCL nanofibers was tested for cytotoxicity in human breast cancer cells. Cancer cells were tested for 72 hours with neat PCL nanofibers and paclitaxel loaded PCL nanofibers in comparison to controlled breast cancer cells. Neat PCL nanofibers showed increased growth while Paclitaxel loaded nanofibers exhibited continuous decrease of number of viable cells. Bright field microscopic images of Prostate cancer (PC3) cells are shown in FIG. 6. Continuous decrease of attached cells (cell density) indicated the slow release of paclitaxel from the NF.

Proliferation or cytotoxicity assay showed significantly decreased proliferation in MDA-MB-231 cells treated with PCL+Paclitaxel, PCL+5FU, and PCL+Paclitaxel+5FU compared to that of untreated and cells treated with PCL only (FIG. 16). Similarly, trypan blue dye exclusion test also confirmed the results of WST assay. Number of dead cell varied among the drug treatments. There were 33% dead cells in PCL+Paclitaxel+5FU treated condition. Both PCL+Paclitaxel and PCL+5FU treated conditions caused cell death between 40% to 50%.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A uniform core/shell nanomaterial comprising:
at least one pharmaceutically active agent;
at least one biocompatible, biodegradable polymer encapsulating the pharmaceutically active agent;
wherein at least one diameter (d) of the nanomaterial has a length no greater than about 1,000 nm, about 900 nm, about 800 nm, about 700 nm, about 600 nm, about 500 nm, about 400 nm, about 300 nm, about 200 nm, or about 100 nm; and
wherein the core contains less than 5% by weight of the biocompatible, biodegradable polymer.

2. The nanomaterial according to claim 1, wherein at least one diameter (d) has a length from about 10-1,000 nm, about 10-900 nm, about 10-800 nm, about 10-700 nm, about 10-600 nm, about 10-500 nm, about 10-400 nm, about 10-300 nm, about 10-200 nm, about 10-100 nm, about 100-1,000 nm, about 100-750 nm, about 100-500 nm, 100-400 nm, 100-300 nm, about 100-250 nm, about 100-200 nm, about 50-200 nm, or about 50-100 nm.

3. The nanomaterial according to claim 1, wherein the diameter (d) has a standard deviation no greater than 25%, 20%, 10%, 5%, 2.5% or 1% of the average diameter (d).

4. The composition according to claim 1, wherein the biocompatible, biodegradable polymer comprises one or more polyester, mixed polyester, polyanhydride, mixed polyanhydride, poly(ester)anhydride, polysaccharide, polyphosphazene or polyphosphoester.

5. The composition according to claim 1 wherein the biocompatible, biodegradable polymer comprises PLGA, polycaprolactone, polylactide, polyglycolide, polyhydroxybutyric acid, poly(sebacic acid), poly[1,6-bis(p-carboxyphenoxy)hexane],

6. The composition according to claim 1, wherein when immersed in 0.01 M PBS (pH 7.4) at 37° C., no more than 5% of the pharmaceutically active agent is released within 24 hours, and at least 75% of the pharmaceutically active agent is released within 56 days.

7. The composition according to claim 1, wherein the pharmaceutically active agent comprises one or more anticancer drug.

8. The composition according to claim 7, wherein the anticancer drug comprises an alkylating agent, an antimetabolite, an anti-microtubule agent, a tyrosine kinase inhibitor, a topoisomerase inhibitor, CSF1R inhibitor, or a cytotoxic antibiotic.

9. The composition according to claim 7, wherein the anticancer drug comprises paclitaxel, gemcitabine, GW 2580, or fluorouracil.

10. The composition according to claim 1, wherein the nanomaterial is in the form of particles, wherein no average diameter is greater than about 2,500 nm, 1,000 nm, 750 nm, 500 nm, 400 nm, 300 nm, 250 nm, 200 nm, 150 nm, or 100 nm.

11. The composition according to claim 10, wherein the pharmaceutically active agent is in nanocrystalline form.

12. The composition according to claim 11, where the pharmaceutically active agent has a particle size less than 200 nm, 175 nm, 150 nm, 125 nm, 100 nm, 75 nm, 50 nm or 25 mm.

13. The composition according to claim 1, further comprising a targeting factor, dye or biomarker.

14. A method for preparing a monodisperse core-shell nanomaterial comprising:
a) providing a core solution comprising at least one pharmaceutically active ingredient;
b) providing a shell solution comprising at least one biocompatible, biodegradable polymer; and
c) coaxial electrospinning the core and shell solutions to form a core-shell nanomaterial.

15. A method for preparing a nanocrystalline drug, comprising:
a) providing a solution of drug in a solvent;
b) electrospraying the solution onto a collector; and
c) removing the solvent to give the nanocrystalline drug.

16. The method according to claim 15 wherein the solvent is removed by vaporization during the electrospraying.

17. The method of claim 15, further comprising the steps:
a) preparing a mixture comprising a nanocrystalline drug, a biocompatible, biodegradable polymer and a water immiscible solvent; and
b) combining said mixture with a water miscible solvent under agitation for a time sufficient to encapsulate the nanocrystalline drug; and
c) removing the water immiscible solvent; and
d) separating encapsulated nanocrystalline drug from the water miscible solvent.

18. The method of claim 17, wherein the water miscible solvent comprises water.
